(19) United States
(12) Patent Application Publication
(10) Pub. No.: US 2001/0003162 A1
(43) Pub. Date: Jun. 7, 2001

(54) Toric intraocular lens materials

(76) Inventors: Kwan Chan, Fort Worth, TX (US); Mutlu Karakelle, Fort Worth, TX (US); Michael J. Simpson, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 09/760,996

(22) Filed: Jan. 16, 2001

Related U.S. Application Data
(63) Continuation-in-part of application No. 09/389,436, filed on Sep. 3, 1999.
Application No. 09/389,436 is a continuation-in-part of application No. 09/283,601, filed on Apr. 1, 1999, now abandoned.
(60) Provisional application No. 60/081,875, filed on Apr. 15, 1998.

(51) Int. Cl. A61F 2/16

(52) U.S. Cl. 623/6.23; 623/6.61

(57) ABSTRACT

A method of selecting an intraocular lens material for toric lenses is disclosed. The method comprises determining the material's Collagen IV Index.

This application is a continuation-in-part application of U.S. patent application Ser. No. 09/389,436, filed Sep. 3, 1999, which is a continuation-in-part application of U.S. patent application Ser. No. 09/283,601, filed Apr. 1, 1999, which claims priority from U.S. Provisional Patent Application No. 60/081,875, filed Apr. 15, 1998.

### FIELD OF THE INVENTION

This invention relates to intraocular lenses. In particular, the present invention relates to toric intraocular lenses.

### BACKGROUND OF THE INVENTION

Foldable intraocular lens (“IOL”) materials can generally be divided into three categories: silicone materials, hydrogel materials, and non-hydrogel acrylic materials. Many materials in each category are known. See, for example, Foldable Intraocular Lenses, Ed. Martin et al., Slack Incorporated, Thorofare, N.J. (1993). Biocompatibility varies among different IOL materials within and among each category. Although the distinction between hydrogel and nonhydrogel acrylic materials is sometimes unclear, for purposes of the present application, acrylic materials that absorb 5% (by weight) or less water at 37° C. are considered non-hydrogel acrylic materials.

One measure of biocompatability for an IOL can be the incidence of posterior capsule opacification (“PCO”). A number or factors may be involved in causing and/or controlling PCO. For example, the design and edge sharpness of an IOL may be a factor. See, Nagamoto et al., J. Cataract Refract. Surg., 23:866-872 (1997); and Nagata et al., Jpn. J. Ophthalmol., 40:397-403 (1996). See, also, U.S. Pat. Nos. 5,549,670 and 5,693,094. Another factor appears to be the lens material itself. See, for example, Mandle, “Acrylic lenses cause less posterior capsule opacification than PMMA, silicone IOLs,” Ocular Surgery News, Vol. 14. No. 15, p. 23 (1996). See, also, Oshika, et al., “Two Year Clinical Study of a Soft Acrylic Intraocular Lens,” J. Cataract. Refract. Surg., 22:104-109 (1996); and Ursell et al., “Relationship Between Intraocular Lens Biomaterials and Posterior Capsule Opacification,” J. Cataract Refract. Surg., 24:352-360 (1998).

One method of addressing the PCO problem involves administering a pharmaceutical agent to the capsular bag area at the time of, or immediately after, extracapsular cataract extraction. See, for example, U.S. Pat. Nos. 5,576,345 (pharmaceutical agent=the cytotoxic agent taxol or an ophthalmically acceptable derivative); 4,515,794; and 5,370,687. Alternatively, the pharmaceutical agent may be tethered to the surface of the IOL material. See, for example, U.S. Pat. No. 4,918,165. The pharmaceutical agents are intended to kill or prevent the growth of proliferating cells that might cause PCO or “secondary cataracts.” Yet another method involves the physical destruction or removal of lens epithelial cells. See, Saika et al., J. Cataract Refract. Surg., 23:1528-1531 (1997).

Another method of addressing PCO is the prophylactic laser therapy method disclosed in U.S. Pat. No. 5,733,276. According to this method, the lens capsule is irradiated with laser irradiation to destroy cells which remain in the lens capsule after extraction of a cataract.

Other methods theorized for reducing the risk of PCO involve adhering the posterior capsule to the IOL at the time of implantation, as in U.S. Pat. No. 5,002,571. According to the '571 patent, a non-biological glue or, preferably, a biological glue, such as fibrin, collagen, or mussel glue, is used to adhere the posterior lens capsule to the posterior surface of an IOL. The glue may be applied over the entire posterior surface of the IOL or just as an annulus around the outer perimeter of the posterior surface of the IOL.

In contrast, U.S. Pat. No. 5,375,611 discloses a method of reducing the risk of PCO by preventing the adherence of the posterior capsule to the IOL. According to the '611 patent, the posterior surface of the lens capsule itself is chemically modified at the time of extracapsular cataract extraction. The chemical modification is achieved by depositing a water-insoluble stable or permanent layer of a cell attachment preventing compound onto the posterior surface of the lens capsule. The stable or permanent layer may be a polymer, such as polyethylene glycol, polysaccharides, polyethylenepropylene glycol, and polyvinyl alcohols.

Aside from biocompatibility concerns, positional stability after implantation is a very important concern for toric IOLs. Toric IOLs are designed to be oriented in a specific way in order to provide the desired vision correction. These IOLs should not rotate or slip from their implanted position.

### SUMMARY OF THE INVENTION

The present invention relates to a method of determining the propensity of an intraocular lens (“IOL”) material to prevent posterior capsule opacification (“PCO”). The method involves incubating replicate samples of an IOL material in a liquid composition comprising collagen IV for a time sufficient to allow at least some of the collagen IV to be adsorbed onto the surface of the IOL material, washing off any loosely bound collagen IV, and then determining for a first sample the amount of collagen IV that remains bound to the IOL material after washing. A second sample is further processed by subjecting it to a collagen IV desorption step and a second washing step. The amount of collagen IV that remains bound to the second sample of IOL material following the desorption and second washing steps is then determined and compared to the amount that remained bound to the first sample. The amount of collagen IV that remains bound after the desorption step can be considered to be specifically or permanently bound, in contrast to any amount of collagen IV that is only non-specifically or transiently bound to the IOL material.

The present invention also relates to IOL materials capable of permanently binding to collagen IV to an extent sufficient to allow an IOL posterior optic surface that contacts the posterior lens capsule to prevent PCO. Without intending to be bound by any theory, it is believed that IOL posterior surfaces that specifically and strongly bind to the lens capsule significantly reduce the risk of or prevent PCO.

The present invention also relates to a method of selecting a material for toric IOLs. IOL materials that bind well to collagen IV allow implanted toric IOLs to remain in their intended position and provide their designed correction.

### BRIEF DESCRIPTION OF THE DRAWINGS

FIG. 1 compares the collagen IV adsorption on ACRYSOF and PMMA materials under different dose/time and washing conditions. Dose refers to the concentration of collagen IV in the liquid composition contacted with the test IOL material. Time refers to the duration of the IOL material's exposure to the liquid composition comprising collagen IV.

FIG. 2 compares the amount of collagen IV remaining adsorbed on various IOL materials following the initial collagen IV adsorption and washing steps to the amount remaining after the surfactant (sodium dodecyl sulfate, “SDS”) desorption and second washing steps.

FIG. 3A shows an edge profile of an ACRYSOF® non-hydrogel acrylic IOL (model MA60BM) optic at a magnification of 150 x.

FIG. 3B shows an edge profile of an ACRYSOFO non-hydrogel acrylic IOL (model MA60BM) optic at a magnification of 150 x with anterior side (up) and posterior side (down) sharp corner angles identified.

FIG. 4A shows an edge profile of a silicone IOL (model SI30NB) optic at a magnification of 150 x.

FIG. 4B shows an edge profile of a silicone IOL (model SI30NB) optic at a magnification of 150 x with anterior and posterior side round corners identified.

FIG. 5 shows an edge profile of a SENSAR® non-hydrogel acrylic IOL (model AR40) optic at a magnification of 150 x.

FIG. 6 shows an edge profile of a HYDROVIEW® hydrogel IOL (model H60M) optic at a magnification of 150 x.

### DETAILED DESCRIPTION OF THE INVENTION

According to the present invention, the propensity of an IOL material to prevent PCO is determined by a method comprising the steps of:

a) incubating a first and second replicate samples of the IOL material in a liquid composition comprising collagen IV at approximately human body temperature for a time sufficient to allow at least some of the collagen IV to adhere to the IOL material;

b) washing any loosely bound collagen IV off of the first and second replicate samples with a washing composition that lacks a collagen IV desorption agent;

c) determining the amount of collagen IV that remains adhered to the first replicate sample;

d) incubating the second replicate sample in a solution comprising a collagen IV desorption agent, wherein the solution has an approximately neutral pH and a temperature of about human body temperature; and

e) washing the second replicate sample in a composition lacking a collagen IV desorption agent; and

f) determining the amount of collagen IV that remains adhered to the second sample and comparing it to the amount of step (c).

The IOL material to be tested according to the method of the present invention is prepared to form samples that can be of almost any size or shape, but are preferably the size and shape of an IOL optic. Two replicate samples of the same IOL material, having approximately identical size and shape, are generally required. (It is possible to use only one sample for the method of the present invention, but using two samples is much more efficient).

As used herein, “collagen IV desorption agent” means an agent selected from the group consisting of (i) hydrophobic agents, such as lipids, and (ii) surfactants.

### Step (a): Collagen IV adsorption step

In the first step of the method of the present invention, each of two replicate samples is incubated in a liquid composition comprising collagen IV at approximately human body temperature for a time sufficient to allow at least some of the collagen IV to adhere to the IOL material.

Human, bovine and rabbit collagen IV, and perhaps other species' collagen IV as well, are commercially available. Human collagen IV is preferred. Collagen IV is usually supplied in the form of a dry powder, but, as in the case of tritium-radio labelled collagen IV, for example, can also be supplied in the form of a solution comprising acetic acid. If obtained in dry powder form, the collagen IV can be dissolved using a diluted weak acid, such as acetic acid. For example, the collagen IV can be dissolved in a 10 μM solution of acetic acid in deionized water. The amount of collagen IV contained in the liquid composition comprising collagen IV will generally be about 2 mg/ml or less, and is preferably about 0.2 mg/ml.

The liquid collagen IV composition should be at approximately neutral pH (about pH 7-7.6) and human body temperature (about 35-37° C.). The liquid collagen IV composition is preferably at pH 7.2-7.4. The liquid collagen IV composition is preferably a buffered salt solution, such as Tris-buffered BSS® or a buffered 0.9% NaCl solution, having an osmolarity approximately equal to that of aqueous humor. The amount of the liquid collagen IV composition comprising collagen IV to be used for each IOL material sample should be that amount sufficient to completely submerse the sample in the liquid composition. The samples are preferably isolated in individual vials, such as plastic microfuge tubes of 1.5-2 ml size, rather than combined in a bath.

The IOL material sample should be incubated in the liquid collagen IV composition for time sufficient to allow at least some of the collagen IV to adhere to the surface of the sample. Depending upon the size and shape of the sample, the identity of the IOL material, the concentration of collagen IV in the liquid collagen IV composition and the amount of the liquid collagen IV composition, etc., the incubation time will generally be about 24 hours or less, preferably about 2-4 hours.

### Step (b): Washing step for replicate samples 1 & 2

After incubating the samples in step (a), the replicate samples are removed from the liquid collagen IV composition and washed extensively using a washing composition comprising a washing agent selected from the group consisting of water, saline and buffered salt solution, in order to remove any loosely bound collagen IV. The washing composition does not contain any collagen IV desorption agent. The washing agent is preferably a buffered salt solution, such as BSSO. The washing is preferably accomplished by soaking the first and second replicate samples in the buffered salt solution for about 30-60 minutes, with the buffered salt solution being replaced with fresh buffered salt solution at about 5-10 minute intervals. This washing step is preferably carried out at a temperature of 20-37° C.

### Step (c): Determining amount of collagen IV adhered to replicate sample 1

After washing in step (b), the amount of collagen IV remaining adhered to the first replicate sample is determined. Suitable methods for determining the amount of collagen IV adhered to the sample include radiolabelling, dye-staining and immunochemical methods. Examples of radiolabelling methods include liquid scintillation counting (e.g., with tritium or ¹⁴C) and gamma isotope counting (e.g., ¹²⁵I) methods. If a radiolabelling method is used, the liquid collagen IV composition of step (a) also comprises radio labelled collagen in an amount of about 2 μCi/ml or less, and preferably about 1 μCi/ml.

### Step (d): Surfactant desorption step

After washing in step (b), the second replicate sample is incubated in a composition comprising a collagen IV desorption agent, wherein the composition has an approximately neutral pH and a temperature of approximately human body temperature. Collagen IV desorption agents include hydrophobic agents, such as lipids, and surfactants. Preferred collagen IV desorption agents are surfactants. Although not essential, the collagen IV desoption agent can be contained in water, saline, or buffered salt solution. For example, the desoption composition can comprise a surfactant in deionized water buffered with 10 mM phosphate buffer. Suitable surfactants include almost any surfactant; it is not essential that the surfactant be nonionic, anionic or cationic. Preferred surfactants include sodium dodecyl sulfate and Triton X-100. In general, the amount of the collagen IV desorption agent contained in the desorption composition will be about 4% (w/v) or less, and preferably about 2% (w/v). The incubation time for this desorption step (step (d)) is generally about 60 minutes or less, and preferably about 15-30 minutes.

### Step (e): Washing step for replicate sample 2

After the second replicate sample has been incubated with a composition comprising a collagen IV desorption agent, the second replicate sample is then washed extensively with a composition lacking a collagen IV desorption agent as described in step (b) above. This washing step removes any residual collagen IV desorption agent and any desorbed collagen IV for the second replicate sample. As in step (b) above, the washing composition may be selected from the group consisting of water, saline and buffered salt solution, but is preferably a buffered salt solution such as BSS®. Again as in step (b), the washing is preferably accomplished by soaking the second sample in buffered salt solution for about 30-60 minutes, with the buffered salt solution being replaced with fresh buffered salt solution at about 5-10 minute intervals. This washing step is preferably carried out at a temperature of 20-37° C.

### Step (f): Determining amount of collagen IV adhered to replicate sample 2

After the second replicate sample has been washed in step (e), the amount of collagen IV remaining adhered to the second replicate sample is determined and compared to the amount adhered to the first replicate sample (determined in step (c)). Suitable methods for determining the amount of collagen IV adhered to the sample include those mentioned above. The amount of collagen IV remaining adhered to the second replicate sample expressed as a percentage of the amount of collagen IV remaining adhered to the first replicate sample is defined as the “Collagen IV Index.”

The method of the present invention can be used to select IOL materials that are capable of reducing the risk of or preventing PCO. Many IOL materials are known, including silicone, hydrogel and foldable non-hydrogel acrylic hydrophobic IOL materials. According to the present invention, IOL materials are screened for their ability to permanently adhere to collagen IV, provided that the IOL materials selected according to the present invention do not consist essentially of (i) 2-phenylethyl methacrylate and 2-phenylethyl acrylate; (ii) ethyl acrylate, ethyl methacrylate and trifluoroethylmethacrylate; or (iii) 2-phenylethyl acrylate and 2-hydroxyethylmethacrylate. IOL materials that have a Collagen IV Index of about 30-100% are preferred. Even more preferred are IOL materials that have a Collagen IV Index of about 50-100%. Most preferred are IOL materials that have a Collagen IV Index of about 75-100%. Suitable IOL materials for screening using the method of the present invention include soft acrylic materials, including but not limited to those disclosed in U.S. Pat. Nos. 5,290,892 and 5,331,073, the entire contents of which are hereby incorporated by reference. The IOL materials of the present invention are used to form IOL bodies or are used to coat all or part of an IOL body. Preferably, at least a portion of the posterior surface of the IOL body comprises the materials of the present invention.

Also preferred are IOL materials which, in addition to having a Collagen IV Index of about 30-100%, are substantially free of glistenings in a physiologic environment. Glistenings are the result of condensation of water vapor within the lens. Although glistenings have no detrimental effect on the function or performance of IOLs made from acrylic materials, it is nevertheless cosmetically desirable to minimize or eliminate them. IOL materials are substantially free of glistenings in a physiologic environment if they have an average of no more than approximately 1-2 glistenings per mm² when evaluated in the test described below. Preferably, the average number of glistenings per mm² will be much less than 1.

The presence of glistenings is measured by placement of a lens sample into a vial and adding deionized water or a balanced salt solution. The vial is then placed into a water bath preheated to 45° C. Samples are to be maintained in the bath for 24 hours. The sample is then placed either in a 37° C. bath or at room temperature and allowed to equilibrate for 2 hours. The sample is removed from the vial and placed on a microscope slide. Visualization of glistenings is done with light microscopy using a magnification of 50 to 200 x.

Preferably, IOL materials are also selected so that they possess the following refractive index, Tg, and elongation properties, which make the materials particularly suitable for use in IOLs which are to be inserted through incisions of 5 mm or less.

The IOL material preferably has a refractive index of at least about 1.50 as measured by an Abbe' refractometer at 589 nm (Na light source). IOL optics made from materials having a refractive index lower than 1.50 are necessarily thicker than optics of the same power which are made from materials having a higher refractive index. As such, IOL optics made from materials having a refractive index lower than about 1.50 generally require relatively larger incisions for IOL implantation.

The glass-transition temperature (“Tg”) of the IOL material, which affects the material's folding and unfolding characteristics, is preferably between about −20 to +25° C., and more preferably between about −5 and +16° C. Tg is measured by differential scanning calorimetry at 10° C./min., and is determined at the midpoint of the transition of the heat flux curve.

The IOL material should also have an elongation of at least about 150%, preferably at least 200%, and most preferably about 300-600%. This property indicates that an IOL optic made of the material generally will not crack, tear or split when folded. Elongation of polymer samples is determined on dumbbell shaped tension test specimens with a 20 mm total length, length in the grip area of 4.88 mm, overall width of 2.49 mm, 0.833 mm width of the narrow section, a fillet radius of 8.83 mm, and a thickness of 0.9 mm. Testing is performed on samples at ambient conditions using an Instron Material Tester (Model No. 4442 or equivalent) with a 50 Netwon load cell. The grip distance is set at 14 mm and a crosshead speed is set at 500 mm/minute and the sample is pulled until failure. The elongation (strain) is reported as a fraction of the displacement at failure to the original grip distance.

The IOL bodies formed of the materials of the present invention or formed of other materials and coated in whole or in part with the materials of the present invention are preferably designed so that at least one of the optic's anterior and posterior surfaces forms a corner where it meets the optic's edge surface such that, at 150 x magnification (of a cross-sectional view), the corner (i) is a sharp corner having an angle from 70-140°, more preferably 80-130°, and most preferably 90-120°, or (ii) is a round corner that has an arc that subtends an angle of 90° or less to the center of a circle having a radius ≦0.025 mm. As used herein, “optic” and “body” are used interchangeably and both mean the central part of the IOL incorporating the image-forming component of the IOL (see the definition of “body” in ISO/FDIS 11979-1:1999 (E)).

Implantable toric IOLs are designed to correct pre-existing corneal astigmatism, typically in patients undergoing cataract surgery. Toric lOLs have one surface (posterior or anterior) that contains a first radius of curvature at one meridian and a second radius of curvature at a second meridian perpendicular to the first. The axis of toric correction on the IOL must be aligned correctly with the astigmatic axis of the corneal astigmatism for optimal results.

IOLs are commonly implanted in the capsular bag after the cataractous lens is removed. Toric lOLs must remain in a specific orientation within the eye in order to achieve the designed correction. Rotation after implantation is a significant concern with toric lOLs. See, for example, Sun et al., Ophthalmology, 107(9):1776-1782 (2000); Patel et al., Ophthalmology, 106(11):2190-2196 (1999); Nguyen, et al., J. Cataract Refract. Surg., 26:1496-1504 (2000); and Ruhswurm, et al., J. Cataract Refract. Surg., 26:1022-1027 (2000).

Toric IOLs can rotate, slip and/or become “decentered” over time due to a variety of factors including capsular fibrosis, wound healing, and, particularly in the case of undersized lenses, normal movement of the eye. Single- and multi-piece toric IOLs of conventional designs made of materials selected according to the present invention will remain in their intended position when implanted in the capsular bag, rotating less than 10°. In the case of multi-piece designs, it is not necessary for both the optic and haptic(s) to consist solely of materials selected according to the present invention. Preferably, at least the optic consists of materials selected according to the present invention, though IOLs where only the haptic(s) consist of materials selected according to the present invention are also within the scope of the present invention. Toric IOLs made of other materials but having the optic's anterior surface, posterior surface, or both, coated with materials selected according to the present invention also will remain in their intended position, rotating less than 10°, and are within the scope of the present invention. Likewise, toric IOLs made of other materials but having the haptic(s) coated with materials selected according to the present invention will remain in their intended position, rotating less than 10°, and are within the scope of the present invention.

Thus, in one embodiment, the present invention relates to toric intraocular lenses comprising an optic having an anterior surface, posterior surface, or both, consisting of (i.e., coated with) a material that has a Collagen IV Index of about 30-100%, provided that said material does not consist essentially of (i) 2-phenylethyl acrylate and 2-phenylethylmethacrylate or (ii) ethyl acrylate, ethyl methacrylate and trifluoroethylmethacrylate. In this embodiment, where the materials of the present invention form a coating on the optic, the coating should be of uniform thickness. In another embodiment, the optic does not comprise materials selected according to the method of the present invention, but the haptic(s) are coated with materials selected according to the present invention. Coatings can be applied using known techniques, including solution and vapor deposition techniques. The coating, whether on the optic or haptic(s), generally will be about 25 μm or less in thickness.

The invention will be further illustrated by the following examples, which are intended to be illustrative, but not limiting.

### EXAMPLES

1. PMMA (polymethylmethacrylate); ACRYSOF (65 wt. % 2-phenylethyl acrylate; 30 wt. % 2-phenylethyl methacrylate; 3.2 wt. % 1,4-butanediol diacrylate; and 1.8 wt. % 2-(3′-methallyl-2′-hydroxy-5′-methyl phenyl) benzotriazole) with (P) and without (NP) Argon plasma gas treatment according to U.S. Pat. No. 5,603,774; ACRYSOF II (80 wt. % 2-phenylethyl acrylate; 15 wt. % 2-hydroxyethylmethacrylate; 3.2 wt. % 1,4-butanediol diacrylate; and 1.8 wt. % 2-(3′-methallyl-2′-hydroxy-5′-methyl phenyl) benzotriazole) with (P) Argon plasma gas treatment according to U.S. Pat. No. 5,603,774; and silicone (SI-30 from Allergan Medical Optic) were analyzed according to the method of the present invention.

The dose (concentration of collagen IV in the liquid composition of step (a)) was varied from 0.2 mg/ml-1 mg/ml. The incubation time for step (a) was also varied from 2-24 hours. The liquid composition of step (a), which was 37° C. and had a pH of 7.4, comprised Tris-buffered BSS® containing human collagen IV (dissolved with the help of acetic acid) and radio labelled (tritium) human collagen IV in an amount of about 1 μCi/ml. The washing of step (b) was accomplished by incubating the samples in 37° C. BSS® for >40 minutes, including replacing the BSS® with fresh BSS® every 5-10 minutes. The desorption step (d) was accomplished by incubating the samples for 30 minutes in a 37° C., pH 7.4 composition comprising 2% (w/v) of sodium dodecyl sulfate. The surfactant composition was buffered with 10 mM phosphate buffer. After the second replicate samples were removed from the surfactant composition, they were washed as in step (b) above.

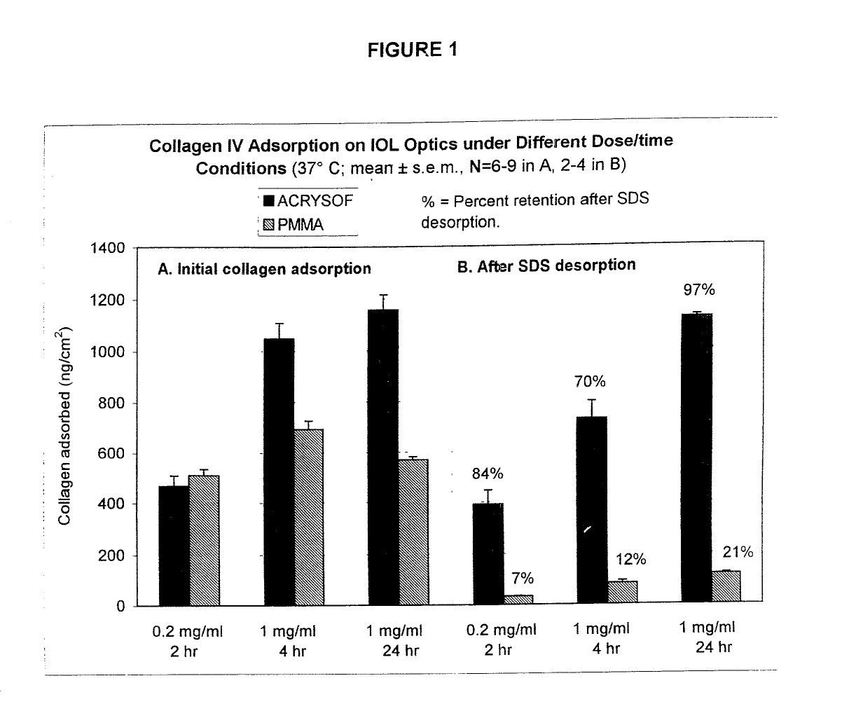

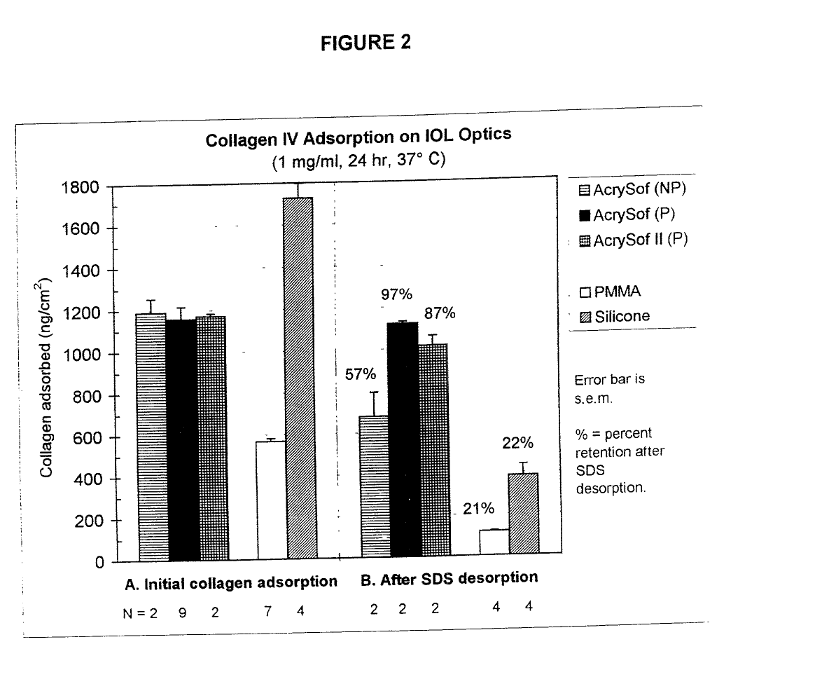

The amount of collagen IV adhered to the samples was determined using a scintillation solution and counted in a P-counter. The data is expressed as amount of collagen IV adsorbed per surface area (ng/cm). Each run consisted of two replicate samples of the IOL material in the shape of an IOL optic. The first replicate sample was subjected to step (a) and the washing step of step (b) and then counted using the e-counter (step (c)). The second replicate sample was subjected to step (a), the washing step of step (b), the desorption step of step (d), the washing step of step (b) again (i.e., step (e)), and then counted using the β-counter (step (f)). The percent retention of collagen IV after SDS desorption is determined by comparing (step (f)) the amount of collagen IV adhered to the second replicate sample to that adhered to the first replicate sample. The results are shown in FIGS. 1 and 2.

2. The edge profile of an IOL body is measured by cutting a cross-sectional slice (0.5 mm thick) of the IOL body along the mid-line. The slice is mounted on its side on a microscope slide to produce a cross-sectional view of the optic under microscope at 150 X magnification. A digital image of the edge profile is recorded by camera and later reproduced on a computer monitor. In general, the corner of the body edge formed with the anterior or posterior body surface is either sharp or round. A sharp corner is defined by the angle (in degrees) between tangents to the body surface (anterior or posterior) and edge surface at the point of their intersection. This angle is measured by placing a pre-calibrated image of a protractor on the corner. A round corner is defined by the arc forming the corner. This arc is measured by fitting different circles of calibrated radius to coincide with the arc. The angle (in degrees) subtended by the arc of best fit at the center of the fitting circle of known radius is measured by protractor.

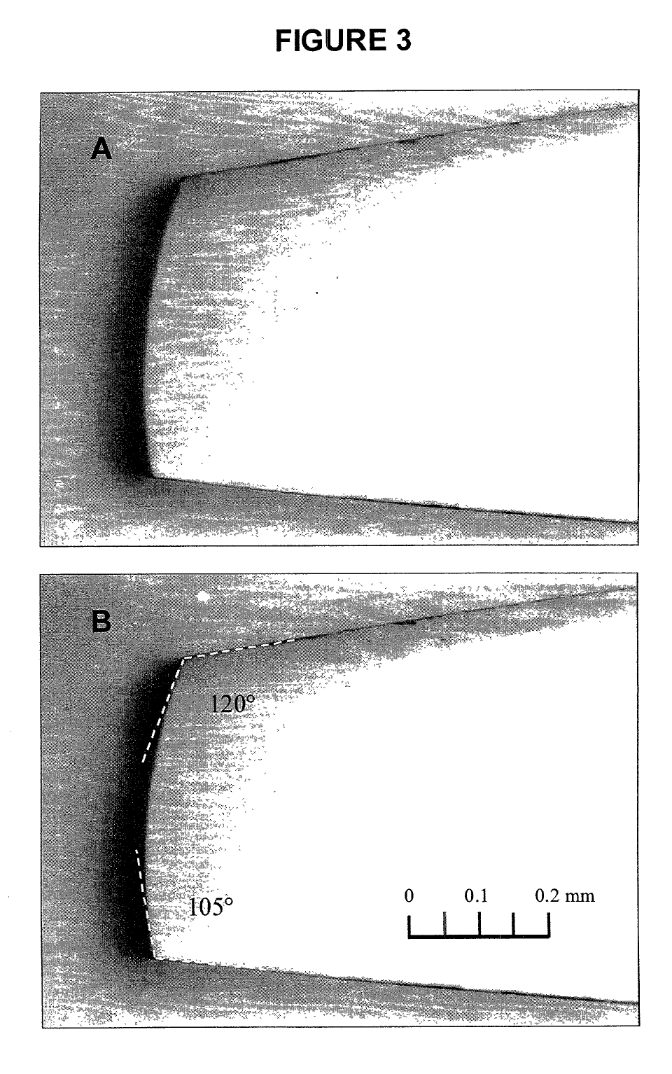

FIG. 3 shows edge profile of ACRYSOF IOL model MA60BM, with the anterior surface of optic facing up in A. In B, the angles of the sharp corners are indicated. The angle between tangents to the optic surface (anterior or posterior) and edge surface at the point of their intersection is measured in degrees.

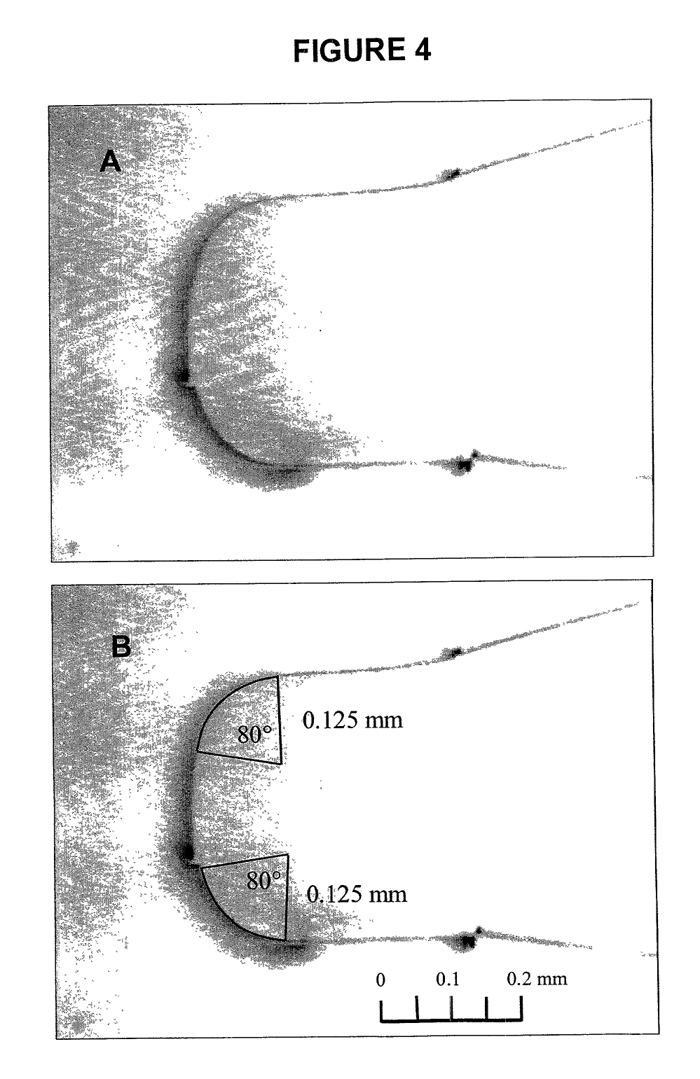

FIG. 4 shows edge profile of silicone IOL model SI30NB in A, and in B the manner with which the arc forming the round corners are measured. The arc of best fit is measured by the angle that the arc subtends at the center of the fitting circle of calibrated radius, in this case 0.125 mm.

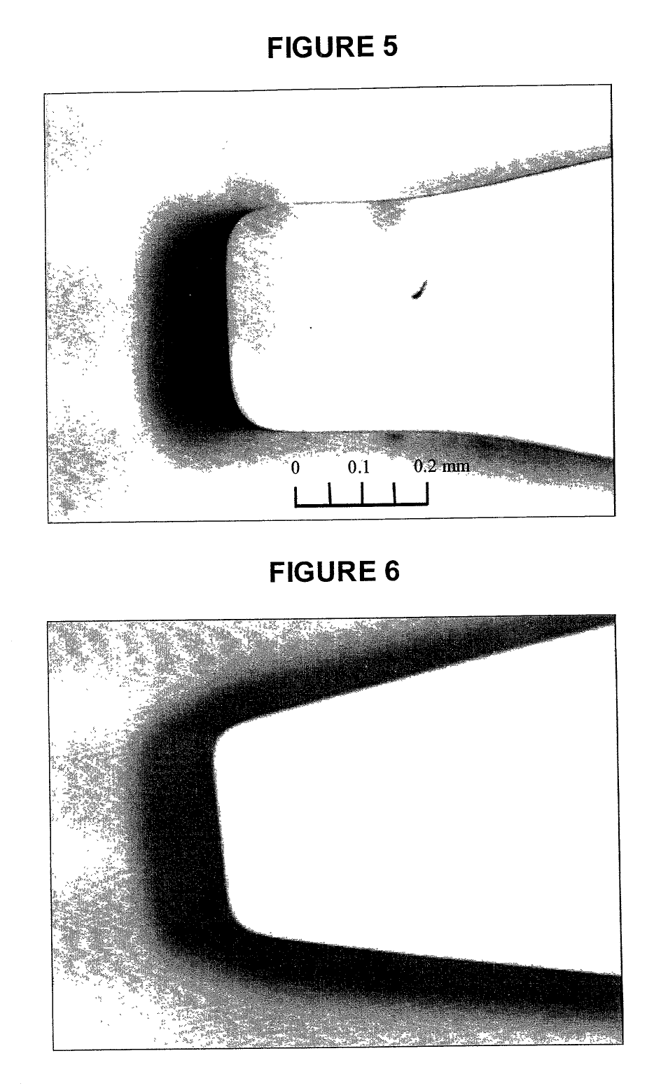

FIGS. 5 and 6 show the edge profiles of acrylic SENSAR®) IOL model AR40 and hydrogel HYDROVIEW® IOL model H60M, respectively. Both IOLs have round corners on the optic edge. The arcs forming the round corners of AR40 are 80° with radius 0.05-0.075 mm, and of H60M are 60-80° with radius of 0.05 mm.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

We claim:

1. A toric intraocular lens comprising an optic having an anterior surface, posterior surface, or both, consisting of a material that has a Collagen IV Index of about 30-100%, provided that said material does not consist essentially of (i) 2-phenylethyl acrylate and 2-phenylethylmethacrylate or (ii) ethyl acrylate, ethyl methacrylate and trifluoroethylmethacrylate.

2. The toric intraocular lens of claim 1 wherein the optic comprises a material that is substantially free of glistenings, has a refractive index of about 1.50 or greater, has a T_{g} of about −20 to +25° C., and has an elongation of at least about 150%.

3. The toric intraocular lens of claim 1 wherein the material has a Collagen IV Index of about 50-100%.

4. The toric intraocular lens of claim 3 wherein the material has a Collagen IV Index of about 75-100%.

5. A toric intraocular lens comprising an optic consisting of a material that has a Collagen IV Index of about 30-100%, provided that said material does not consist essentially of (i) 2-phenylethyl acrylate and 2-phenylethylmethacrylate or (ii) ethyl acrylate, ethyl methacrylate and trifluoroethylmethacrylate.

6. A toric intraocular lens comprising a haptic consisting of a material that has a Collagen IV Index of about 30-100%, provided that said material does not consist essentially of (i) 2-phenylethyl acrylate and 2-phenylethylmethacrylate or (ii) ethyl acrylate, ethyl methacrylate and trifluoroethylmethacrylate.

7. A toric intraocular lens comprising a haptic coated with a material that has a Collagen IV Index of about 30-100%, provided that said material does not consist essentially of (i) 2-phenylethyl acrylate and 2-phenylethylmethacrylate or (ii) ethyl acrylate, ethyl methacrylate and trifluoroethylmethacrylate.
